SEQUENCE LISTING (6 sequences)

SEQ ID NO: 1 - protein, 451 aa, Cornus florida
MDGRHSRISVLMFPWLAHGHISPFLELAKKLTKRNFHIYFCSTPVNLRPIKEKLPEKYSLSIQLVELHLPSLPELPPHYHTTNGLPPHLMPTLKKAFDMASPNFSTIVKNLRPDLLIYDFLQQWAPSIALSRNIPAVEFLPTNAAMTSFVIHLTKNPGVEFPFPEIYLRDNYAISKFQNVLESSANGIKDTERAVECCEQSSEIVLIKTFREIEAKYLDYLSELTGKKIVPVGPLVQDPVHEYDEKPGIMEWLNNKERSSTVFVSFGSEYFLSKEEMEEIAYGLEQSMVPFIWVVRFPGGAKVSLEMALPKGFLDRVGDGGMVVEGWAPQTKILEHSSIGGFSSHCGWSSVLESMKLGVPIIAMPMHLDQPINARLVEEVGMGLEVERDMNGKLKREEVAKVIREVVVEKAGESVRQKAKELKEKLISKGEEEIDEVVQEVVQLCRKKNRH

SEQ ID NO: 2 - protein, 480 aa, Juglans regia
MLITNPTTKISSANTRDRLSSYLLQTTTERERERELLMEAKKNTISILMVPWLAHGHISPFLELAKKLTNRNFHIYMCSTPVNLSSIKKNVTEKYSQSIKLVEIHLPSLPNLPPHYHTTNGLPHHLISTLKTAFEMSTPNFSKILQTLNPDLVIYDFNLPWAADCASSVNIPAVQFLTFSAAVVALGIHMYDKPGEMFPFPEIYLHEYEMLTIKKALEYLPGNKFPFDEALRRSRDIILVKTCRDFEGKYMDYLSSLVSKKVVPVGALVQESIDQDGHEEIMQWLDKKETSSTVFVSFGSEYFLSKEKIHAVAQGLELSKVNFILVIRFPQGERISTQDALPEGFLERIGERGMILEGWAPQAKILQHSSIGGFVSHCGWNSLMESMKFGVPIIAMPMQVDQPMNARLVEYIGIGMEAMRDEKGNLQSEEIAKVIRKVVVDEMGKAVRKNARELSEKMNAKGDEKIDGVVEELVALCNNK

SEQ ID NO: 3 - DNA, 1356 bp, Cornus florida
  source 1..1356: mol_type = genomic DNA
atggatggaaggcacagtagaatatcagtacttatgtttccgtggctggcgcatggtcatatttctccgttcctggagctggcgaagaagttgaccaaacgcaacttccacatttacttctgcagcacaccggtgaacctgcgtccgatcaaggagaaactcccggaaaagtacagcctttccatccagctggtcgagctgcacctgccgagcctgccagaattgccaccccactatcataccaccaatggcctgccgcctcacctgatgccgacgctgaagaaagcctttgatatggcttccccgaatttcagcaccattgtgaaaaacttacgtccggatcttctgatttatgacttcttacaacagtgggcgcctagcatcgcgctgagtcgtaacattccggcagttgagttcttgccgacgaacgccgcaatgacctcgttcgtgatccatttgacgaagaacccgggtgtggaatttccgttcccggaaatctacctgcgtgacaactatgccatttccaaattccagaatgttttggagagctctgcaaatggcataaaggataccgaacgcgcagttgaatgttgtgaacagtctagcgagatcgtgttgatcaaaacctttcgtgagatcgaagcaaagtacctggattatctgtccgagttgactggtaaaaagatcgtgccggttggtccgctggttcaagacccggtgcatgaatacgacgagaaaccgggtattatggaatggttgaataacaaagagagaagctcgaccgtttttgttagctttggcagcgagtactttctgtcgaaggaggagatggaagaaattgcgtatggcttggagcaaagcatggttccgttcatctgggttgtgcgctttccaggtggcgcgaaagttagcctggagatggccctgccgaaaggcttcctggaccgcgtgggcgatggtggcatggtggtcgagggttgggcgccacagaccaaaatcctggagcacagctcgatcggcggtttttcctcccactgcggttggagctcagtactggagtcaatgaaactgggtgttccgatcattgctatgccgatgcacctggaccagccgattaacgctcgtctggttgaggaggtgggcatgggtctcgaggtcgaacgtgacatgaacggcaaattaaagcgcgaagaggttgcgaaggtcattcgcgaagttgttgtagagaaggcgggtgagtccgtgcgtcagaaagcgaaggagttaaaagaaaagctgatcagcaagggtgaagaagaaattgatgaagtggtgcaagaagtcgtacaactgtgccgtaaaaagaatcgtcattaa

SEQ ID NO: 4 - DNA, 1443 bp, Juglans regia
  source 1..1443: mol_type = genomic DNA
atgctaataacaaatcccactacgaaaatttctagcgcaaatacccgtgaccgtttgtcttcatatctgctgcaaaccacgacggaacgcgagcgcgaacgcgagctcctcatggaagcgaaaaagaacaccattagcatcttgatggttccgtggctggcgcatggtcacattagcccgtttctcgagctggcgaagaagctgacgaaccgtaatttccatatttatatgtgcagcactccggtgaacttatctagcattaaaaaaaatgttaccgaaaagtatagccagtccatcaaactggtcgagatccatctgccaagcttgcccaatctgcctccgcactaccataccaccaacggtctgccgcaccacctgatttctacgctgaaaactgccttcgagatgagtaccccgaacttctctaagatcctgcaaaccctgaatccggatctggtcatctacgacttcaacctgccgtgggcagctgattgtgcgagcagcgttaacattccggcagtgcagtttttgacctttagcgcggcggtggtggccctgggtatccacatgtatgacaaaccgggtgaaatgtttccgttcccggagatctacctgcatgagtatgaaatgctgaccattaagaaggcgctggagtacctgccgggcaacaaattcccgtttgatgaagcgctgcgtcgtagccgtgacatcatcttggtgaaaacctgtcgtgactttgaaggcaaatacatggattatctgtcctctttggtcagcaagaaagtggtgccggttggtgctctggttcaagaaagcattgatcaggacggccacgaagaaattatgcaatggttggataagaaagagacaagcagcaccgtttttgtgtcgttcggcagcgagtacttcttgagcaaagagaagatccacgccgtggcccagggtttagaacttagtaaggtgaacttcatcctggtgatccgctttccgcagggcgaacgtatttccacccaggatgcattgccggagggcttcttggaacgcatcggtgaacgtggtatgattctggagggttgggcaccgcaggctaagatcttacaacatagctccatcggcggttttgtttctcactgcggttggaacagcctgatggagagcatgaaattcggtgttccgattatcgccatgccgatgcaggttgaccagccgatgaacgctcgtctggtcgagtacattggcatcggcatggaagctatgcgtgatgagaagggtaatctacaatccgaagagattgcgaaggtgatccgcaaagttgtggtagacgagatgggtaaagcggttcgtaaaaacgcgagagaactctccgagaaaatgaatgcgaagggcgacgagaagatcgacggcgttgtagaggaactcgttgcgctttgcaacaacaaataa

SEQ ID NO: 5 - DNA, 20 bp, synthetic construct
  source 1..20: mol_type = other DNA
taatacgactcactataggg

SEQ ID NO: 6 - DNA, 20 bp, synthetic construct
  source 1..20: mol_type = other DNA
tgctagttattgctcagcgg

(19) United States
(12) Patent Application Publication
(10) Pub. No.: US 2026/0110007 A1
(43) Pub. Date: Apr. 23, 2026

(54) GLUCOSYLTRANSFERASE CATALYZING REBAUDIOSIDE A TO PRODUCE REBAUDIOSIDE M AND USE THEREOF

(71) Applicant: Shandong Benyue Biological Technology Co., Ltd, Dongying (CN)

(72) Inventors: Shunbin ZHANG, Dongying (CN); Liang CHEN, Dongying (CN); Zhen ZHANG, Dongying (CN); Jinpeng JIAO, Dongying (CN); Boqing DING, Dongying (CN)

(73) Assignee: Shandong Benyue Biological Technology Co., Ltd, Dongying (CN)

(21) Appl. No.: 19/359,758

(22) Filed: Oct. 16, 2025

(30) Foreign Application Priority Data
Oct. 22, 2024 (CN) 202411471320.5

(51) Int. Cl. C12P 19/04; C12N 9/10; C12N 15/70; C12P 19/18

(52) U.S. Cl. CPC C12P 19/04; C12N 9/1051; C12N 9/1062; C12N 15/70; C12P 19/18; C12Y 204/01013; C12N 2800/101; C12N 2830/002

(57) ABSTRACT

A glucosyltransferase catalyzing RA to produce RM and a use thereof are provided. The glucosyltransferases derived from Cornus florida and Juglans regia L. are screened and obtained. It was found that the glucosyltransferase derived from the Cornus florida exhibited a relatively high enzymatic activity in catalyzing the RA to produce the RD, while the glucosyltransferase derived from the Juglans regia L. exhibited a relatively high enzymatic activity in catalyzing the RD to produce the RM. When these two enzymes were used together in catalyzing the RA to produce the RM, a relatively high conversion rate was achieved. The use of the glucosyltransferases derived from the Cornus florida and the Juglans regia L. for preparing the RM of SGs, has not been reported yet. The glucosyltransferase provides an excellent candidate protein resource and its corresponding gene sequence for the enzymatic conversion process of the SGs.

### CROSS REFERENCE TO THE RELATED APPLICATIONS

This application is based upon and claims priority to Chinese Patent Application No. 202411471320.5, filed on Oct. 22, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBFSTZB031-PKG_SequenceListing.xml, created on Sep. 29, 2025, and is 9,896 bytes in size.

### TECHNICAL FIELD

The invention belongs to the technical field of enzyme engineering, and specifically relates to a glucosyltransferase catalyzing rebaudioside A (RA) to produce rebaudioside M (RM) and a use thereof.

### BACKGROUND

Steviol glycosides (SGs) include stevioside, RA, rebaudioside B (RB), rebaudioside C (RC), rebaudioside D (RD), rebaudioside E (RE), rebaudioside F (RF), dulcoside A, steviolbioside, RM, and rebaudioside I (RI). RM is formed by connecting the R1 end of the RD structure to the glucosyl group via a β-1,3 glycosidic bond. The content of RM in Stevia rebaudiana is less than 0.1%, much lower than RA and stevioside. However, compared with RA and stevioside, RM has a high sweetness (about 300-350 times the sweetness of sucrose), rapid sweetness release, and pure sweetness without off-flavors such as bitterness, astringency, sourness, and licorice flavor. Its high sweetness and good taste make it a new generation of sweeteners. As a sucrose substitute, RM has shown significant potential for application in the food industry, effectively reducing reliance on traditional sweeteners, thereby alleviating the environmental burden of their production, including land use, water resources consumption, and greenhouse gas emissions. In this way, the use of RM contributes, to a certain extent, to maintaining the balance and health of the entire ecosystem, particularly by playing a positive role in protecting our marine environment.

The glucosyltransferases involved in RM synthesis mainly include three enzymes: β-1,2-glucosyltransferase, which converts RA to RD; β-1,3-glucosyltransferase, which converts RD to RM; and sucrose synthase, which is involved in the glycosyl cycle. As a key factor influencing the efficiency of RM synthesis, the diversity of glucosyltransferase sources and species is particularly important. During the RM synthesis process, different enzymes may have different catalytic efficiency, substrate specificity, and stability. If different types of glucosyltransferases can be obtained from multiple sources, it may be possible to find one or more enzymes that can catalyze the RM synthesis reaction more efficiently and specifically. However, the currently known enzyme types are relatively limited, which compels us to continuously explore and develop new enzyme resources to further improve the efficiency of RM synthesis.

### SUMMARY

The technical problem to be solved by the present invention is to provide a glucosyltransferase catalyzing the RA to produce the RM and a use thereof. The enzyme exhibits relatively high activity, can directionally transfer glucose groups to produce the RM, and achieve a relatively high conversion rate.

The technical solution of the present invention is as follows:

The glucosyltransferase catalyzing the RA to produce the RM of the present invention includes a glucosyltransferase derived from Cornus florida and a glucosyltransferase derived from Juglans regia L. The amino acid sequence of the glucosyltransferase derived from the Cornus florida is shown as SEQ ID NO: 1, and the amino acid sequence of the glucosyltransferase derived from the Juglans regia L. is shown as SEQ ID NO: 2.

The nucleotide sequence of the glucosyltransferase derived from the Cornus florida is shown as SEQ ID NO: 3, and the nucleotide sequence of the glucosyltransferase derived from the Juglans regia L. is shown as SEQ ID NO: 4.

The amino acid sequence of the glucosyltransferase derived from the Cornus florida is
shown as SEQ ID NO: 1:
MDGRHSRISVLMFPWLAHGHISPFLELAKKLTKRNFHIYFCSTPVNLRPIKEKLP
EKYSLSIQLVELHLPSLPELPPHYHTTNGLPPHLMPTLKKAFDMASPNFSTIVKNLRPDLL
IYDFLQQWAPSIALSRNIPAVEFLPTNAAMTSFVIHLTKNPGVEFPFPEIYLRDNYAISKFQ
NVLESSANGIKDTERAVECCEQSSEIVLIKTFREIEAKYLDYLSELTGKKIVPVGPLVQDPV
HEYDEKPGIMEWLNNKERSSTVFVSFGSEYFLSKEEMEEIAYGLEQSMVPFIWVVRFPG
GAKVSLEMALPKGFLDRVGDGGMVVEGWAPQTKILEHSSIGGFSSHCGWSSVLESMKL
GVPIIAMPMHLDQPINARLVEEVGMGLEVERDMNGKLKREEVAKVIREVVVEKAGESV
RQKAKELKEKLISKGEEEIDEVVQEVVQLCRKKNRH.
The amino acid sequence of the glucosyltransferase derived from the Juglans regia L. is
shown as SEQ ID NO: 2:
MLITNPTTKISSANTRDRLSSYLLQTTTERERERELLMEAKKNTISILMVPWLAH
GHISPFLELAKKLTNRNFHIYMCSTPVNLSSIKKNVTEKYSQSIKLVEIHLPSLPNLPPHYH
TTNGLPHHLISTLKTAFEMSTPNFSKILQTLNPDLVIYDFNLPWAADCASSVNIPAVQFLTF
SAAVVALGIHMYDKPGEMFPFPEIYLHEYEMLTIKKALEYLPGNKFPFDEALRRSRDIILV
KTCRDFEGKYMDYLSSLVSKKVVPVGALVQESIDQDGHEEIMQWLDKKETSSTVFVSFG
SEYFLSKEKIHAVAQGLELSKVNFILVIRFPQGERISTQDALPEGFLERIGERGMILEGWAP
QAKILQHSSIGGFVSHCGWNSLMESMKFGVPIIAMPMQVDQPMNARLVEYIGIGMEAM
RDEKGNLQSEEIAKVIRKVVVDEMGKAVRKNARELSEKMNAKGDEKIDGVVEELVALCNNK.
The nucleotide sequence of the glucosyltransferase derived from the Cornus florida is
shown as SEQ ID NO: 3:
atggatggaaggcacagtagaatatcagtacttatgtttccgtggctggcgcatggtcatatttctccgttcctggagctggcgaa
gaagttgaccaaacgcaacttccacatttacttctgcagcacaccggtgaacctgcgtccgatcaaggagaaactcccggaaaagtacagc
ctttccatccagctggtcgagctgcacctgccgagcctgccagaattgccaccccactatcataccaccaatggcctgccgcctcacctgat
gccgacgctgaagaaagcctttgatatggcttccccgaatttcagcaccattgtgaaaaacttacgtccggatcttctgatttatgacttcttaca
acagtgggcgcctagcatcgcgctgagtcgtaacattccggcagttgagttcttgccgacgaacgccgcaatgacctcgttcgtgatccattt
gacgaagaacccgggtgtggaatttccgttcccggaaatctacctgcgtgacaactatgccatttccaaattccagaatgttttggagagctct
gcaaatggcataaaggataccgaacgcgcagttgaatgttgtgaacagtctagcgagatcgtgttgatcaaaacctttcgtgagatcgaagc
aaagtacctggattatctgtccgagttgactggtaaaaagatcgtgccggttggtccgctggttcaagacccggtgcatgaatacgacgaga
aaccgggtattatggaatggttgaataacaaagagagaagctcgaccgtttttgttagctttggcagcgagtactttctgtcgaaggaggaga
tggaagaaattgcgtatggcttggagcaaagcatggttccgttcatctgggttgtgcgctttccaggtggcgcgaaagttagcctggagatg
gccctgccgaaaggcttcctggaccgcgtgggcgatggtggcatggtggtcgagggttgggcgccacagaccaaaatcctggagcaca
gctcgatcggcggtttttcctcccactgcggttggagctcagtactggagtcaatgaaactgggtgttccgatcattgctatgccgatgcacct
ggaccagccgattaacgctcgtctggttgaggaggtgggcatgggtctcgaggtcgaacgtgacatgaacggcaaattaaagcgcgaag
aggttgcgaaggtcattcgcgaagttgttgtagagaaggcgggtgagtccgtgcgtcagaaagcgaaggagttaaaagaaaagctgatca
gcaagggtgaagaagaaattgatgaagtggtgcaagaagtcgtacaactgtgccgtaaaaagaatcgtcattaa.
The nucleotide sequence of the glucosyltransferase derived from the Juglans regia L. is
shown as SEQ ID NO: 4:
atgctaataacaaatcccactacgaaaatttctagcgcaaatacccgtgaccgtttgtcttcatatctgctgcaaaccacgacgga
acgcgagcgcgaacgcgagctcctcatggaagcgaaaaagaacaccattagcatcttgatggttccgtggctggcgcatggtcacattag
cccgtttctcgagctggcgaagaagctgacgaaccgtaatttccatatttatatgtgcagcactccggtgaacttatctagcattaaaaaaaatg
ttaccgaaaagtatagccagtccatcaaactggtcgagatccatctgccaagcttgcccaatctgcctccgcactaccataccaccaacggtc
tgccgcaccacctgatttctacgctgaaaactgccttcgagatgagtaccccgaacttctctaagatcctgcaaaccctgaatccggatctggt
catctacgacttcaacctgccgtgggcagctgattgtgcgagcagcgttaacattccggcagtgcagtttttgacctttagcgcggcggtggt
ggccctgggtatccacatgtatgacaaaccgggtgaaatgtttccgttcccggagatctacctgcatgagtatgaaatgctgaccattaagaa
ggcgctggagtacctgccgggcaacaaattcccgtttgatgaagcgctgcgtcgtagccgtgacatcatcttggtgaaaacctgtcgtgact
ttgaaggcaaatacatggattatctgtcctctttggtcagcaagaaagtggtgccggttggtgctctggttcaagaaagcattgatcaggacg
gccacgaagaaattatgcaatggttggataagaaagagacaagcagcaccgtttttgtgtcgttcggcagcgagtacttcttgagcaaagag
aagatccacgccgtggcccagggtttagaacttagtaaggtgaacttcatcctggtgatccgctttccgcagggcgaacgtatttccacccag
gatgcattgccggagggcttcttggaacgcatcggtgaacgtggtatgattctggagggttgggcaccgcaggctaagatcttacaacatag
ctccatcggcggttttgtttctcactgcggttggaacagcctgatggagagcatgaaattcggtgttccgattatcgccatgccgatgcaggtt
gaccagccgatgaacgctcgtctggtcgagtacattggcatcggcatggaagctatgcgtgatgagaagggtaatctacaatccgaagaga
ttgcgaaggtgatccgcaaagttgtggtagacgagatgggtaaagcggttcgtaaaaacgcgagagaactctccgagaaaatgaatgcga
agggcgacgagaagatcgacggcgttgtagaggaactcgttgcgctttgcaacaacaaataa.

The present invention provides a use of the glucosyltransferase with the amino acid sequence shown as SEQ ID NO: 1 and the glucosyltransferase with the amino acid sequence shown as SEQ ID NO: 2 in catalyzing the RA to produce the RM.

The glucosyltransferase with the amino acid sequence shown as SEQ ID NO: 1 is β-1,2-uridine-5′-diphosphate (UDP)-glucosyltransferase, and the glucosyltransferase with the amino acid sequence shown as SEQ ID NO: 2 is β-1,3-UDP-glucosyltransferase. The β-1,2-UDP-glucosyltransferase uses the RA as a substrate and, through a cycle established by the sucrose synthase, produces the RD; and the β-1,3-UDP-glucosyltransferase uses the RD as a substrate and, through the cycle established by the sucrose synthase, produces the RM.

A method for catalyzing the RA to produce the RM includes the following steps:
(1) preparing an enzyme solution of the glucosyltransferase with the amino acid sequence shown as SEQ ID NO: 1 and an enzyme solution of the glucosyltransferase with the amino acid sequence shown as SEQ ID NO: 2, respectively; and
(2) using the RA as a substrate, adding sucrose and sucrose synthase, then adding the enzyme solutions prepared in the step (1), and performing a catalytic reaction to obtain the RM. Catalytic reaction conditions are as follows: a pH value is 6-7, and a temperature is 37-38° C. Addition amounts of the RA, the sucrose, the sucrose synthase, and the enzyme solutions can be adjusted according to actual needs.

In the step (1), the method for preparing the enzyme solutions includes the following steps:
a, constructing an expression vector carrying a glucosyltransferase gene, where the expression vector is a pET vector, preferably pET-28a(+);
b, transforming the expression vector into a host cell to obtain a recombinant strain, where the host cell includes Escherichia coli, preferably E. coli BL21 (DE3) or E. coli Rosetta (DE3), more preferably E. coli BL21 (DE3); and
c, inducing expression in the recombinant strain at 16-25° C. for 16-36 h, using isopropyl-β-d-thiogalactoside (IPTG) as an inducer, collecting bacterial cells, lysing the bacterial cells by low-temperature ultrasonication, and obtaining a supernatant as the enzyme solution.

Compared with the prior art, the present invention possesses the following advantages:

The present invention screened and obtained glucosyltransferases derived from the Cornus florida and the Juglans regia L. It was found that the glucosyltransferase derived from the Cornus florida exhibited a relatively high enzymatic activity in catalyzing the RA to produce the RD, while the glucosyltransferase derived from the Juglans regia L. exhibited a relatively high enzymatic activity in catalyzing the RD to produce the RM. When these two enzymes were used together in catalyzing the RA to produce the RM, a relatively high conversion rate was achieved. The use of the glucosyltransferases derived from the Cornus florida and the Juglans regia L. for preparing the RM of SGs has not been reported yet. The present invention provides an excellent candidate protein resource and its corresponding gene sequence for the enzymatic conversion process of the SGs, enhances the application potential of glucosyltransferases in the food, pharmaceutical, and chemical industries, and has important implications for the industrial production of glucosyltransferases.

### BRIEF DESCRIPTION OF THE DRAWINGS

FIG. 1 shows the high-performance liquid chromatography (HPLC) chromatogram of enzyme activity of glucosyltransferase derived from Cornus florida;

FIG. 2 shows the HPLC chromatogram of enzyme activity of glucosyltransferase derived from Juglans regia L.;

FIG. 3 shows the HPLC chromatogram of the product catalyzed by the enzyme solution of the glucosyltransferases derived from Cornus florida and Juglans regia L.

### DETAILED DESCRIPTION OF THE EMBODIMENTS

The examples of the present invention are intended only to further illustrate the content of the present invention and are not intended to limit its content or scope. For molecular biology experimental methods not specifically described in the present examples, please refer to Molecular Cloning: A Laboratory Manual.

The culture media used in the examples are as follows:
Luria-Bertani (LB) medium: 10 g/L tryptone, 5 g/L yeast extract, 10 g/L sodium chloride; and
Terrific Broth (TB) medium: 12 g/L tryptone, 24 g/L yeast extract, 5 g/L glycerol, 2.31 g/L KH₂PO₄, 16.43 g/L K₂HPO₄·3H₂O.

### Example 1: Construction of Glucosyltransferase Strains

Expression vectors carrying glucosyltransferase genes were constructed. The glucosyltransferases were derived from Cornus florida, Nyssa sinensis Oliv., Camellia japonica L., Actinidia chinensis, Juglans regia L., Solanum bulbocastanum, Solanum dulcamara L., and Nicotiana attenuata.

The expression vector was transformed into host E. coli BL21 (DE3) cells, then plated onto LB plates containing 50 mg/L kanamycin sulfate and cultured overnight at 37° C. Single colonies were picked from the plates and verified by polymerase chain reaction (PCR) using universal primers. Positive clones were retained to obtain recombinant strains.

The recombinant strain was inoculated into LB liquid medium at the 1% inoculum to prepare the seed culture. This seed culture was then inoculated into TB liquid medium at the 1% inoculum, and induced and cultured in the shaking incubator at 25° C. (with 1 mM IPTG as the inducer) for 24 h. The fermentation broth was centrifuged at 4° C. and 12,000 rpm for 15 min. The bacterial pellet was collected and then lysed by low-temperature sonication, and the supernatant was collected to obtain the enzyme solution.

The nucleotide sequence of the forward primer is:
5′-TAATACGACTCACTATAGGG-3′,
as shown in SEQ ID NO: 5;
The nucleotide sequence of the reverse primer is:
5′-TGCTAGTTATTGCTCAGCGG-3′,
as shown in SEQ ID NO: 6.

PCR reaction system: 1 μL each of the 10 μM forward and reverse primers, 25 μL of the 2×PCR mix, 1 μL of the template, and adding the double-distilled water to bring the final volume to 50 μL.

PCR reaction procedure: 94° C. pre-denaturation for 4 min; followed by 25 cycles (94° C. for 10 s, 55° C. for 5 s, and 72° C. for 7 min 50 s); extension at 72° C. for 10 min; and hold at 4° C. at the end.

PCR products were verified by 1% agarose gel electrophoresis.

### Example 2 Glucosyltransferase Enzyme Activity Assay

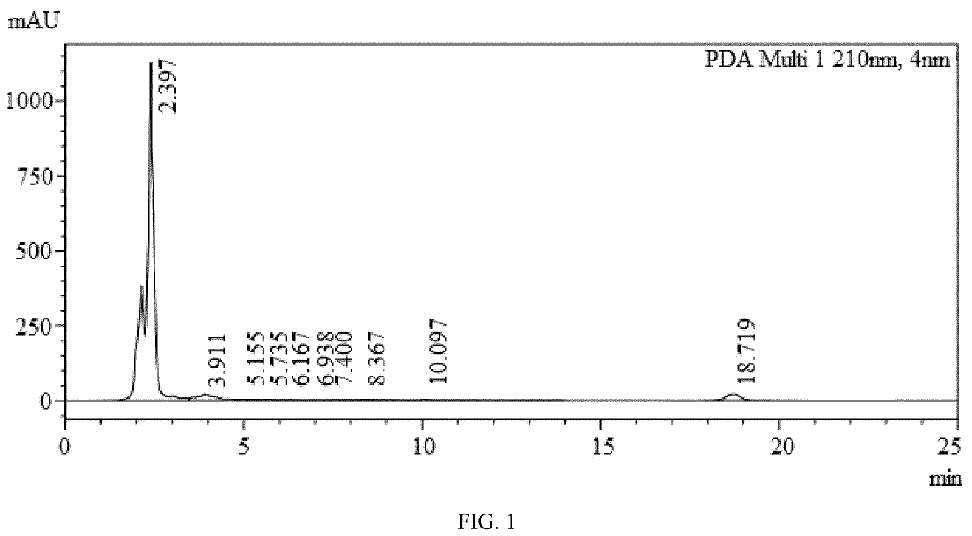

The enzyme solutions of glucosyltransferases derived from Cornus florida, Nyssa sinensis Oliv., Camellia japonica L., Actinidia chinensis, Juglans regia L. prepared in Example 1 were subjected to the enzyme activity assays of the RA-RD catalytic reaction. The enzyme activity assay method was as follows: 1 mL of the 1.2 mM RA, 1 mL of the uridine diphosphoglucose (UDPG), and 1 mL of the enzyme solution were reacted at 37° C., 200 rpm for 1 h, followed by HPLC assay. The results are shown in Table 1. The HPLC chromatogram of enzyme activity of the glucosyltransferase derived from Cornus florida is shown in FIG. 1.

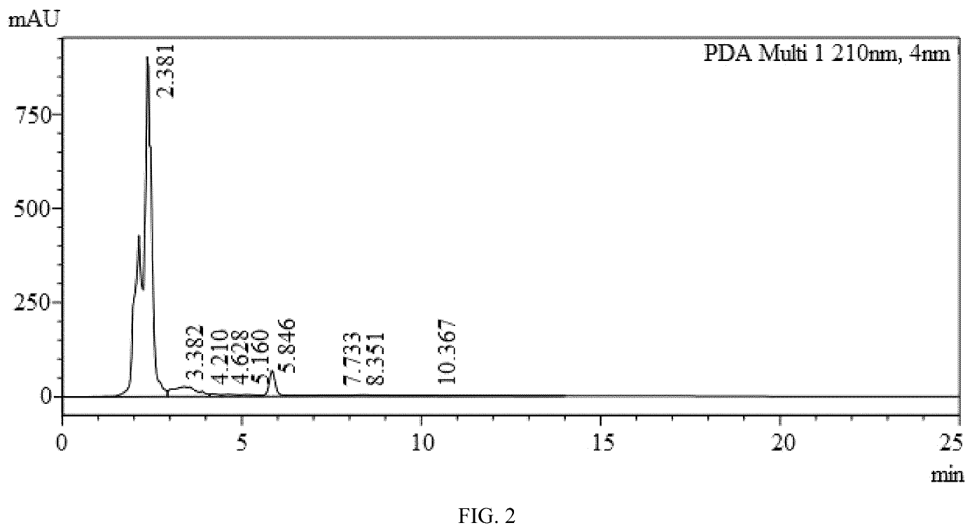

The enzyme solutions of glucosyltransferases obtained in Example 1 derived from Solanum bulbocastanum, Solanum dulcamara L., Nicotiana attenuata, Cornus florida, and Juglans regia L. were subjected to the enzyme activity assays of the RD-RM catalytic reaction. The enzyme activity assay method was as follows: 1 mL of the 1.2 mM RD, 1 mL of the UDPG, and 1 mL of the enzyme solution were reacted at 37° C., 200 rpm for 1 h, followed by HPLC assay. The results are shown in Table 2. The HPLC chromatogram of enzyme activity of the glucosyltransferase derived from Juglans regia L. is shown in FIG. 2.

The specific HPLC assay method parameters are as follows: adding 200 μL of chromatography-grade acetonitrile, shaking to mix well, letting stand for 10 min, centrifuging at 12,000 rpm for 10 min at room temperature, and passing the supernatant through the 0.22 μm organic membrane for analysis. The HPLC was performed using the C18 reverse-phase bonded silica gel separation column (4.6 mm×250 mm, 5 μm) with 25% acetonitrile as the mobile phase at the flow rate of 1 mL/min and the column temperature of 40° C., the UV detector photodiode array (PDA) was used at the PDA detector wavelength of 210 nm, and the injection volume was 10 μL.

TABLE 1
Enzyme activity assay results of RA-RD catalytic reaction

| Sample Name | Retention Time (min) | Peak Area |
|---|---|---|
| Cornus florida | 5.735 | 82819 |
| Nyssa sinensis Oliv. | 5.734 | 9858 |
| Camellia japonica L. | 5.741 | 7724 |
| Actinidia chinensis | 5.754 | 9815 |
| Juglans regia L. | 5.742 | 6959 |

TABLE 2
Enzyme activity assay results of RD-RM catalytic reaction

| Sample Name | Retention Time (min) | Peak Area |
|---|---|---|
| Solanum bulbocastanum | 7.799 | 39018 |
| Solanum dulcamara L. | 7.756 | 40278 |
| Nicotiana attenuata | 7.765 | 44516 |
| Cornus florida | 7.767 | 56501 |
| Juglans regia L. | 7.733 | 207717 |

As shown in Tables 1 and 2, the enzymatic activity of the glucosyltransferase derived from Cornus florida was significantly higher than that of the enzymes derived from other sources in the RA-RD catalytic reaction, while the enzymatic activity of the glucosyltransferase derived from Juglans regia L. was significantly higher than that of the enzymes derived from other sources in the RD-RM catalytic reaction. Therefore, the glucosyltransferases derived from Cornus florida and Juglans regia L. can be used for the directed transfer of glucosyl groups to catalyze the RA to produce the RM of the SGs.

### Example 3: Conversion of Glucosyltransferases

The enzyme solutions of the glucosyltransferases derived from Cornus florida and Juglans regia L. prepared in Example 1 were used in the catalytic reaction: water was used as the solvent, along with 20 g/L RA, 300 g/L sucrose, 50 g/L sucrose synthase, 100 g/L enzyme solution of the glucosyltransferase derived from Cornus florida, and 150 g/L enzyme solution of the glucosyltransferase derived from Juglans regia L. The catalytic reaction was carried out at 37° C. and pH 7 for 24 h.

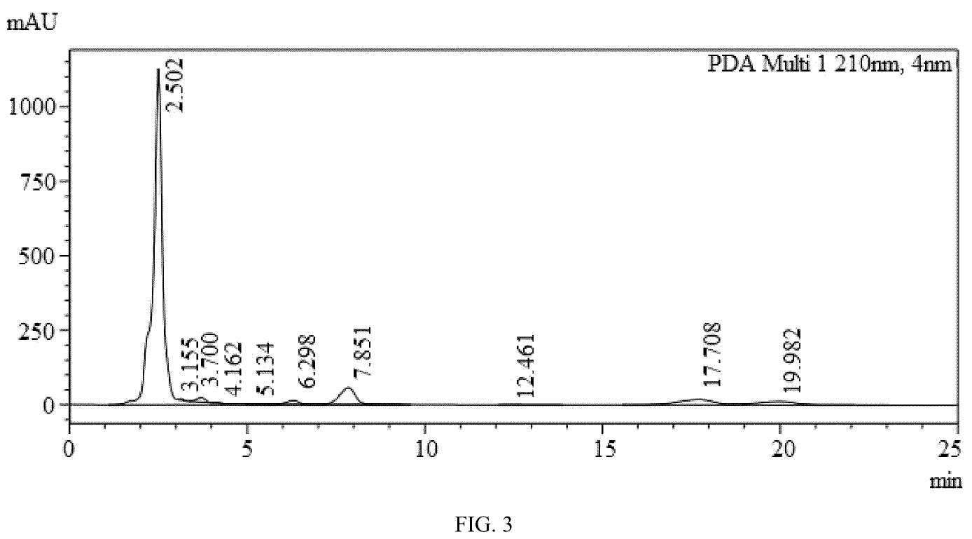

After the reaction, HPLC was used to detect the product formation. The results are shown in FIG. 3. The glucosyltransferases derived from Cornus florida and Juglans regia L. can catalyze the RA to produce the RM by directed transfer of glucose groups. After 24 h of reaction, 10 g/L RM was produced.

1. A glucosyltransferase catalyzing rebaudioside A (RA) to produce rebaudioside M (RM), wherein the glucosyltransferase comprises a glucosyltransferase derived from Cornus florida and a glucosyltransferase derived from Juglans regia L., the amino acid sequence of the glucosyltransferase derived from the Cornus florida is shown as SEQ ID NO: 1, and the amino acid sequence of the glucosyltransferase derived from the Juglans regia L. is shown as SEQ ID NO: 2.

2. The glucosyltransferase catalyzing the RA to produce the RM according to claim 1, wherein the nucleotide sequence of the glucosyltransferase derived from the Cornus florida is shown as SEQ ID NO: 3, and the nucleotide sequence of the glucosyltransferase derived from the Juglans regia L. is shown as SEQ ID NO: 4.

3. A use of the glucosyltransferase catalyzing the RA to produce the RM according to claim 1, wherein the glucosyltransferase with the amino acid sequence shown as SEQ ID NO: 1 and the glucosyltransferase with the amino acid sequence shown as SEQ ID NO: 2 are used to catalyze the RA to produce the RM.

4. The use according to claim 3, wherein a method for catalyzing the RA to produce the RM comprises the following steps:
(1) preparing an enzyme solution of the glucosyltransferase with the amino acid sequence shown as SEQ ID NO: 1 and an enzyme solution of the glucosyltransferase with the amino acid sequence shown as SEQ ID NO: 2, respectively; and
(2) using the RA as a substrate, adding sucrose and sucrose synthase, then adding the enzyme solutions prepared in the step (1), and performing a catalytic reaction to obtain the RM.

5. The use according to claim 4, wherein a method for preparing the enzyme solution comprises the following steps:
a, constructing an expression vector carrying a glucosyltransferase gene;
b, transforming the expression vector into a host cell to obtain a recombinant strain; and
c, after inducing expression in the recombinant strain, collecting bacterial cells, lysing the bacterial cells by low-temperature ultrasonication, and obtaining a supernatant as the enzyme solution.

6. The use according to claim 5, wherein the expression vector is a pET vector, and the host cell comprises Escherichia coli.

7. The use according to claim 5, wherein an inducer used for the inducing expression is isopropyl-β-d-thiogalactoside.

8. The use according to claim 4, wherein catalytic reaction conditions in the step (2) are as follows: a pH value is 6-7, and a temperature is 37-38° C.
